(19) United States
(12) Patent Application Publication
(10) Pub. No.: US 2004/0267305 A1
(43) Pub. Date: Dec. 30, 2004

(54) Flexible tube contents remover

(76) Inventor: Mark H. Borgman, Windsor, CO (US)

Correspondence Address: ANCEL W. LEWIS, JR., 425 WEST MULBERRY, SUITE 101, FORT COLLINS, CO, 80521, US

(21) Appl. No.: 10/609,852

(22) Filed: Jun. 30, 2003

(51) Int. Cl. A61B 17/00; A61B 17/28

(52) U.S. Cl. 606/209; 606/1

(57) ABSTRACT

Flexible tube contents removers that are particular suitable for a variety of medical procedures include spaced arms supporting a pair of parallel spaced tapered rollers having spacing that is adjustable through finger pressure. One embodiment has the flexible tube extending through a hole in one support arm that directs the tube between the rollers. Another embodiment includes the rollers at an angle to a line normal to the longitudinal axis of the support arm so the tube extends along one side of the support arms and between the rollers. Stop surfaces restrict movement of the arms beyond a selected acute inside angle. A cam surface and resilient cut-out section on the arms serves as a bias to return the arms to a position of maximum spacing for the rollers when the finger pressure is removed.

### FIELD OF THE INVENTION

This invention relates to devices to remove the contents from inside a flexible tube and more particularly to a flexible tube contents remover that is useful for a variety of medical procedures.

### DESCRIPTION OF THE PRIOR ART

There are a number of medical procedures that use a flexible tube to drain contents taken from a patient. These medical procedures include a thoracostomy, pneumothorax, hemothorax, hemopneumothorax, tension pneumothorax, empyema, chylothorax, pleural effusion and post-operative cardiothoracic surgery.

Cardiac tamponade may occur following cardiac surgery as a result of blood or fluid collecting in the pericardial space compressing the heart and reducing cardiac output. Mediastinal chest drains (including pericardial drains) are inserted as standard post-operative practice following cardiac surgery to assist the clearance of blood from the pericardial space and to prevent cardiac tamponade. Manipulation techniques including milking and stripping may be applied to the tubes to keep them from blocking. The present invention may also be applied to urinary drainage tubes, feeding tubes, blood tubes and wound drainage tubes.

A number of hand held devices have heretofore been provided using a pair of opposed rollers that squeeze a flexible tube when moved along a tube to remove contents from the tube. U.S. Pat. Nos. 3,648,701, 4,164,223 and 4,819,636 disclosed devices using a pair of opposed rollers on support arms that pivot about on an axis at outer ends to allow the rollers to move toward and away from one another. U.S. Pat. Nos. 3,194,452; 4,287,890 and 4,452,244 disclose devices using a pair of opposed rollers on support arms with an inside pivot axis between the ends of the support arms.

### DISCLOSURE OF THE INVENTION

Flexible tube contents removers disclosed have two support arms pivotally connected at adjacent ends and arranged along an acute inside angle with stop means on the arms to limit the pivotal movement to not moving beyond an acute inside angle. Two rollers are supported on the support arms opposite the pivotal connection that are parallel spaced and move between a range of spaced positions. A flexible tube extends between the rollers and is compressed by the rollers when finger pressure is applied to the support arms and the support arms and rollers are moved along the tube to remove the tubes contents. In one embodiment the tube extends through a hole in one of the support arms so the tube is carried by the support arm. In another embodiment the support arms are modified so the rollers are turned at an angle to a line normal to the longitudinal axis of the arms so the tube extends along one side of the support arms and between the rollers.

### BRIEF DESCRIPTION OF THE DRAWINGS,

FIG. 1 is a front perspective view of a tube contents remover in an open at rest position with a flexible tube carried therein embodying features of the present invention.

FIG. 2 is a rear perspective view of the remover and tube shown in FIG. 1 with a human body and collection bag also shown.

FIG. 3 is a front elevation view of the remover and tube shown in FIGS. 1 and 2.

FIG. 4 is a top plan view of the remover and tube shown in FIGS. 1-3.

FIG. 5 is a sectional view taken along line 5-5 of FIG. 4.

FIG. 6 is a rear perspective view of the remover and tube but inverted from that shown in FIGS. 1-4.

FIG. 7 is a sectional view like FIG. 5 with the rollers in a tube compressing second position.

FIG. 8 is a front perspective view of a second embodiment of a tube contents remover and tube embodying features of the present invention.

FIG. 9 is a rear perspective view of the remover and tube shown in FIG. 8 with a human body and collection bag also shown.

FIG. 10 is a front elevation view of the remover and tube shown in FIGS. 8 and 9.

FIG. 11 is a top plan view of the remover and tube shown in FIGS. 8-10.

FIG. 12 is a sectional view taken along line 12-12 of FIG. 11.

FIG. 13 is a rear perspective view of the remover and tube but inverted from that shown in FIGS. 8-11.

FIG. 14 is a sectional view like FIG. 12 with the rollers in a tube compressing second position.

### DETAILED DESCRIPTION OF THE INVENTION

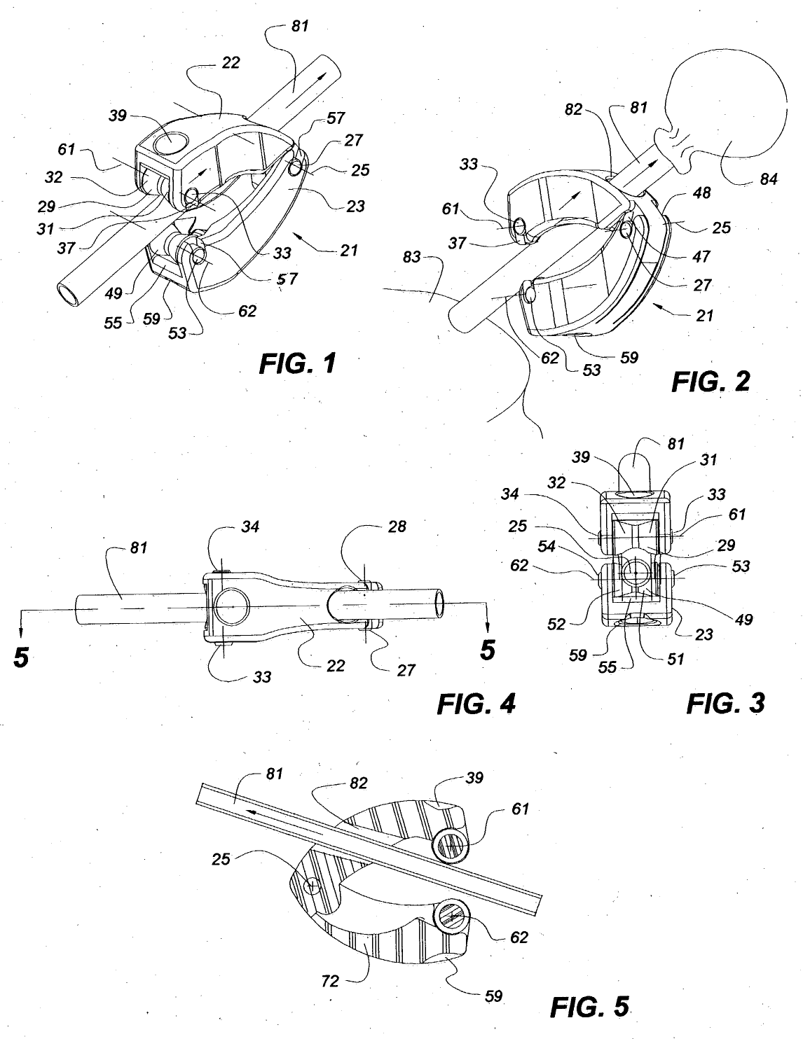

Referring now to FIGS. 1-7, there is shown a tube contents remover 21 which includes a first su pot arm 22 and a second support arm 23 pivotally connected at adjacent respective first end portions so as to move relative to one another about a pivot axis 25. The first support arm 22 is in the form of a curved or arcuate body having a pair of stub shafts 27 and 28 extending transversely of the body away from one another at the rear end along the pivot axis 25. The body is preferably made as an integral, one piece molded plastic unit. A first roller 29 supported on the first support arm 22 is tapered having a pair of tapered peripheral surfaces 31 and 32 decreasing in size from opposite ends toward a midpoint between the opposite ends. The first roller 29 has opposed shaft end portions 33 and 34. These shaft end portions 33 and 34 are received in a channel-shaped recess 35 in a front end portion of first support arm 22 and has slots 37 and 38 in side walls defining the recess into which the shaft end portions 33 and 34 are slidably received and retained so that the rollers 29 will rotate freely relative to the support arm 22. A finger recess 39 is provided in the top surface of the support arm at the front end.

The second support arm 23 is in the form of a curved or arcuate body having a pair of opposed shaft sockets 47 and 48 that receive and support stub shafts 27 and 28, respectively above described to pivotally connect the two support arms 22 and 23 together. The body is preferably made as an integral one piece molded plastic unit. A second roller 49 supported in the second support arm 23 is tapered having a pair of tapered peripheral surfaces 51 and 52 decreasing in size from opposite ends toward a midpoint between the opposite ends. The second roller 49 has opposed shaft end portions 53 and 54 and is received in a channel-shaped recess' 55 in a front portion of the body with slots 57 and 58 in the side wall portions defining the recess 55 with the shaft end portions 53 and 54 slidably received in and rotated relative to the second support arm 23. A finger recess 59 is provided in the bottom surface of the second support arm 23 at the front end.

The first roller 29 rotates about a roller axis 61 and the second roller 49 rotates about roller axis 62. These axes are arranged parallel to one another and are spaced from one another for movement of the first and second rollers 29 and 49 toward and away from one another between a first position and a range of second positions of different spacings between the rollers through the users finger pressure.

The first support arm 22 has a stop surface 68 at the rear end and the second support arm 23 has a stop surface 69 at the rear end with these stop surfaces contacting one another to limit the pivotal movement from moving beyond a selected acute inside angle between the two support arms.

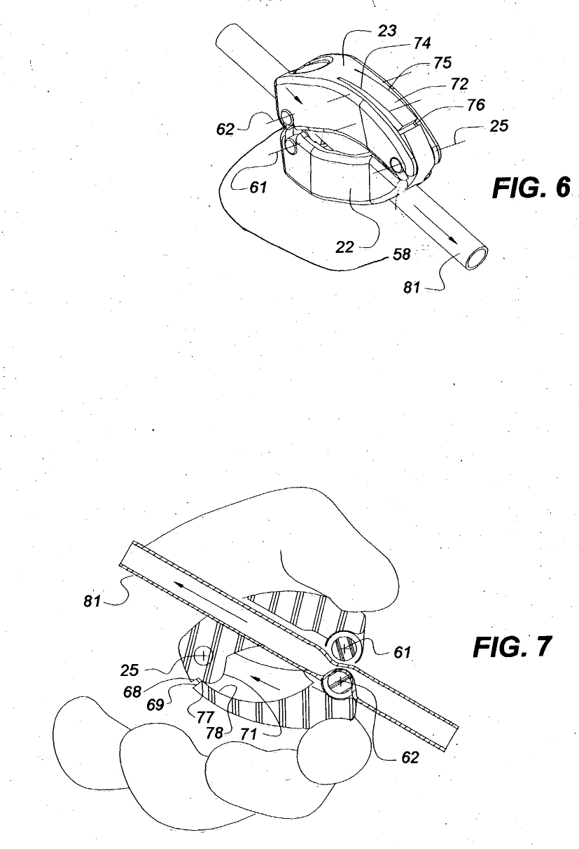

The first support arm 22 has a cam with a cam surface 71 at the rear end and the second support arm has a cut-out section 72 shown in FIG. 6. The cut-out section 72 is formed by cutting along parallel spaced side cut lines to form sides 74 and 75 spaced inwardly from the sides and cutting along an end cut line to form and end 76 spaced inwardly from the front end that forms stop surface 69 as above discussed. The cut-out section is attached opposite end 76. A corner 77 is formed by stop surface 69 and an inner curved surface 78 of the second support arm that rides against the cam surface 71 to raise the cut-out section as the support arms are separated from the first position and the resilience or flexibility of the cut-out section 72 causes the support arms to return to the first position when the finger pressure is removed. The cam surface 71 is formed along a selected radius that will raise or lift the cut-out section 72 and put it under tension in the second position. When the hand pressure is removed the cut-out section 72 slides along the cam surface 71 and acts as a spring to automatically return the arms to the first position of maximum spacing between the rollers as shown in FIG. 5. A flexible tube 81 extends through a hole 82 in the first support arm 22 to allow the tube to extend between the two rollers and have the tube carried by the first support arm 22.

In use, the users finger pressure is applied to the support arms as shown in FIG. 7 to bring the rollers to a second position wherein the rollers squeeze or compress the tube and the rollers are moved along the tube as shown by an arrow so the contents in the tube are removed. Typically one end of the tube extends into the human body 83 and the other connects to a receptacle or collection bag 84 with the device moved by the user away from the human body 83 toward the receptacle 84 to discharge the contents of the tube into the receptacle 84.

The tapered rollers as compared to flat rollers provide tracking to enable the remover to be rolled down the tube without becoming derailed. The taper enables the user to adjust for sub-atmospheric negative pressure that is present in prior as flat roller techniques involving stripping or milking. The tapered rollers provide some degree of suction volume since the tube is not completely closed off in removing an obstruction in the tube. This makes the procedure less traumatic to the pleural lung tissue when the tube is a thoracostomy tube. The ability of the remover to carry the tube as above described is of benefit to the user as the assembly is ready when the physician requests that the nurse remove blockage by milking the chest tube.

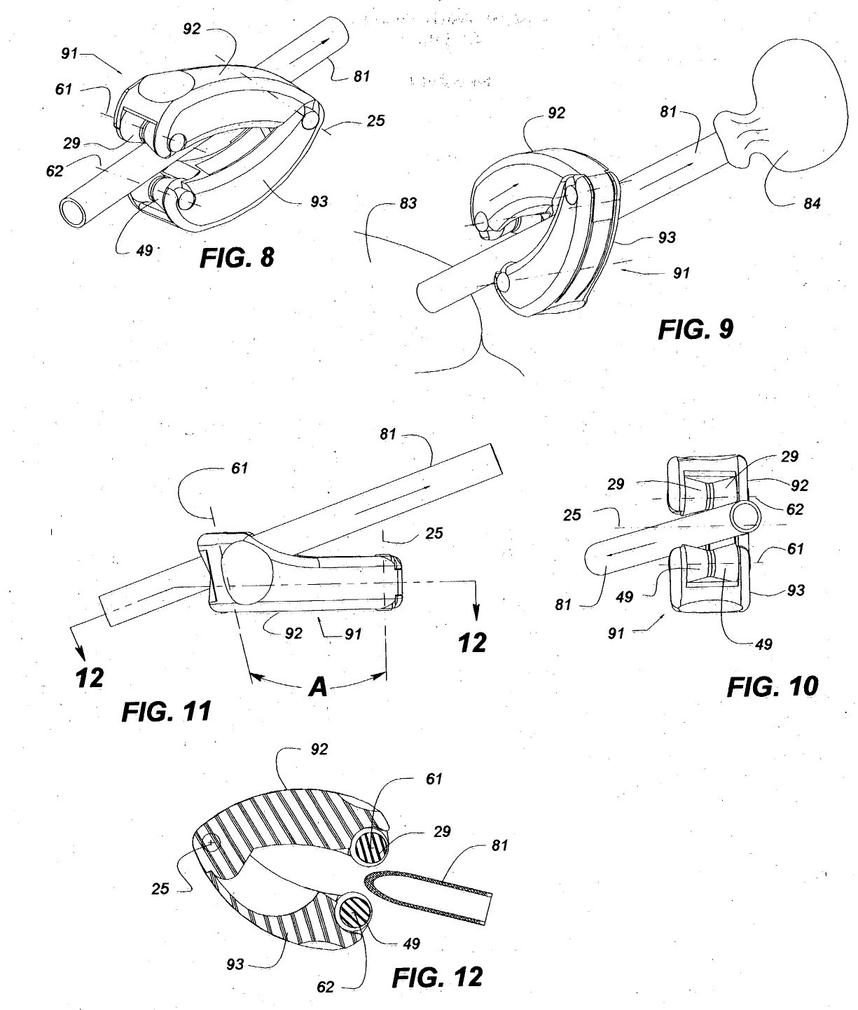

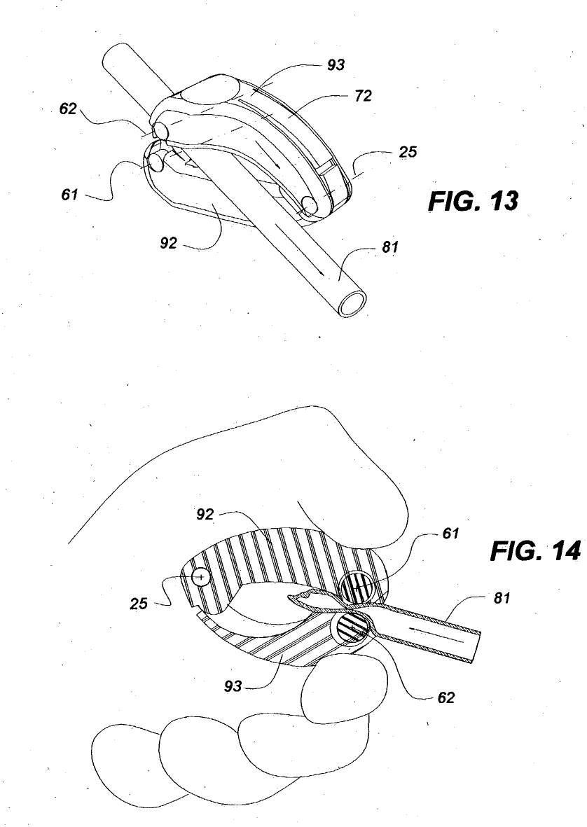

Referring now the FIGS. 8-14, there is shown a another embodiment of a remover 91 which includes first and second support arms 92 and 93 pivotally connected at adjacent respective first end portions to move relative to one another about pivot axis 25 in a manner similar to arms 22 and 23 above described.

The construction of the remover 91 is identical to remover 21 above described as it applies to the support arms supporting first and second rollers 29 and 49 and the operation thereof so the description of remover 21 applies to remover 91. In this embodiment hole 82 is not present.

The first and second rollers 29 and 49 are turned at a slight angle designated A from a line at right angles or normal to the longitude axis of the support arms and the tube extends from one side of the support arms into the remover 91 and between the rollers. The operation of the remover 91 is the same as remover 21 above described.

Although the present invention has been described with a certain degree of particularity, it is understood that the present disclosure has been made by way of example and that changes in details of structure may be made without departing from the spirit thereof.

What is claimed is:

1. A flexible tube contents remover comprising:
first and second support arms pivotally connected at adjacent, respective first end portions to move relative to one another about a pivot axis,
first and second rollers supported on said first and second support arms, respectively, at respective second end portions for rotation about a first roller axis and a second roller axis, respectively, said first and second roller axes being arranged parallel to and spaced from one another for movement of said first and second rollers toward and away from one another between an at rest first position and a range of second positions of different spacings between said rollers adjustable through finger pressure for a flexible tube disposed between said first and second rollers to be compressed when said rollers are in one of said second positions as said rollers and support arms are moved along said tube,
each of said rollers having a peripheral surface decreasing in size from opposite ends toward a midpoint between said opposite ends to provide a gap between said rollers that increases in size from said opposite ends to said midpoint,
whereby a users hand may grip said support arms between fingers and apply pressure to said support arms to move said rollers to a second position for moving said rollers against said tube containing a fluid along the length of said tube to compress said tube and remove contents from inside said tube.

2. The remover as set forth in claim 1 wherein each of said support arms has a stop surface that contacts one another to limit said pivotal movement to a select acute inside angle between said support arms.

3. The remover as set forth in claim 1 when said support arms have biasing means to return said support arms to said first position when said finger pressure is removed.

4. The remover as set forth in claim 3 wherein said biasing means includes a cam on one of said support arms and a flexible cut-out section on one of said support arms that moves over said cam.

5. The remover as set forth in claim 4 wherein said cam has a cam surface arranged along a radius that will raise an end portion of said cut-out section when said arms are in said second position.

6. The remover as set forth in claim 1 wherein each of said support arms are of an arcuate shape.

7. The remover as set forth in claim 1 wherein said support arms have finger depressions for locating opposed pressing fingers of a user on said support arms.

8. The remover as set forth in claim 1 wherein one of said support arms has a hole arranged transverse to said first and second roller axes through which said tube extends to carry said tube and direct said tube between said rollers.

9. The remover as set forth in claim 1 wherein said first and second rollers are turned at an acute angle to a line normal the longitated axis of said support arms to enable said tube to extend along a side of each of said support arms and between said first and second rollers.

10. The remover as set forth in claim 1 wherein said support arms are made as a one piece molded plastic body.

11. A flexible tube contents remover comprising:
first and second support arms pivotally connected at adjacent, respective, first end portions to move relative to one another about a pivot axis,
first and second rollers supported on said first and second support arms, respectively, at respective second end portions for rotation about a first roller axis and a second roller axis, respectively, said first and second roller axes being arranged parallel to and spaced from one another for movement of said first and second rollers toward and away from one another between an at rest first portion and a range of second positions of different spacings between said rollers adjustable through finger pressure for a flexible tube disposed between said first and second rollers to be compressed when said rollers are in one of said second positions as said rollers are move along said tube,
said first support arm having a hole arranged transverse to said first and second roller axes of through which said tube extends to carry said tube and direct said tube between said rollers,
whereby a users hand may grip said support arms between fingers and apply pressure to said support arms to move said rollers to one of said second positions for moving said first and second rollers against said tube along the length of said tube to compress said tube and remove contents from inside said tube.

12. A flexible tube contents remover comprising:
first and second support arms pivotally connect at adjacent, respective, first end portions to move relative to one another about a pivot axis,
first and second rollers supported on said first and second support arms, respectively, at respective second end portions for rotation about a first roller axis and a second roller axis, respectively, said first and second roller axes being arranged parallel to and spaced from one another for movement of said first and second rollers toward and away from one another between an at rest first portion and a range of second positions of different spacings between said rollers adjustable through finger pressure for a flexible tube disposed between said first and second rollers to be compressed when said rollers in one of said second positions as said rollers are move along said tube,
said first and second rollers being turned at an acute angle to a line normal to the longitudinal axis of said support arms to enable said tube to extend along a side of each of said support arms and between said first and second rollers.
